(19) United States
(12) Patent Application Publication
(10) Pub. No.: US 2012/0045505 A1
(43) Pub. Date: Feb. 23, 2012

(54) FIXED DOSE DRUG COMBINATION FORMULATIONS

(75) Inventors: Badal Kumar Sasmal, Hyderabad (IN); Praveen Reddy Billa, Hyderabad (IN); Vijay Dinanathji Nasare, Nagpur (IN); Mailatur Sivaraman Mohan, Hyderabad (IN); Suryakumar Jayanthi, Hyderabad (IN); Rajesh Dubey, Secunderabad (IN); Seshasayana Avvaru, Prakasam (IN); Phanikumar Reddy Satti, West Godavary District (IN); Ranjan Mund Manas, Dist. Kalahandi (IN); Anitha Chidipothu, Hyderabad (IN); Varun Jain, Mainpuri (IN)

(73) Assignees: Dr. Reddy's Laboratories, Inc., Bridgewater, NJ (US); Dr. Reddy's Laboratories Ltd., Hyderabad (IN)

(21) Appl. No.: 13/283,747

(22) Filed: Oct. 28, 2011

Related U.S. Application Data
(63) Continuation of application No. PCT/US2010/033097, filed on Apr. 30, 2010.
Application No. PCT/US2010/033097 is a continuation-in-part of application No. 10/898,549, filed on Jul. 28, 2004.
(60) Provisional application No. 61/256,570, filed on Oct. 30, 2009.

(30) Foreign Application Priority Data
Apr. 30, 2009 (IN) 1015/CHE/2009

(51) Int. Cl. A61K 9/48; A61P 9/04; A61P 9/00; A61P 9/10; A61K 31/616; A61P 29/00

(52) U.S. Cl. 424/451; 514/161

(57) ABSTRACT

Pharmaceutical formulations comprising multiple drugs, for treating or preventing cardiovascular disease. Embodiments are capsules containing individual drugs, or combinations of drugs, in the form of small tablets.

### INTRODUCTION

Aspects of the present invention relate to pharmaceutical compositions having fixed dose combinations of active agents. Aspects also provide processes for preparation of the compositions.

Aspects of the present invention further relate to the management of subjects having an elevated risk of cardiovascular events, cerebrovascular diseases and other associated diseases or disorders. For example, embodiments of the present invention provide fixed dose pharmaceutical formulations for such treatment that combine at least one cholesterol-lowering agent, at least one inhibitor of the renin-angiotensin system, aspirin, and a beta-adrenergic receptor blocking agent or a diuretic, in a single dosage form.

Cardiovascular diseases have been a leading cause of morbidity and mortality worldwide, possibly being responsible for 16.6 million deaths in 2001. The majority (80 percent) of all deaths attributable to cardiovascular diseases (CVDs) are in low- and middle-income countries. By 2010, CVDs are expected to become the leading cause of mortality in developing countries. There is now a pressing need for developing and implementing preventive interventions for effective management of CVDs.

The risk of having a cardiovascular event is not restricted to those with hypertension or hypercholesterolemia, but is continuous down to at least a blood pressure of 115/75 mm Hg and total cholesterol level of 4 mmol/L (about 155 mg/dl). However, it is only in the last few years that clinical trials have confirmed the benefits of blood pressure and cholesterol lowering in high-risk patients who do not have a clinical diagnosis of hypertension or hypercholesterolemia. Hence, the large majority of adults, and virtually all people with established vascular disease, would benefit from blood pressure and cholesterol lowering therapy, which would require simultaneous administration of blood pressure-reducing and cholesterol-lowering agents.

In a recent World Health Organization-Wellcome Trust meeting report, authors outlined the vast unmet needs in cardiovascular therapy and recommended the development of combination products for the same. The number of high-risk individuals who could benefit from affordable combination cardiovascular therapy in India and many other countries is substantial. In a combination product, each component causes a proportional risk reduction which is unaffected by the presence or absence of the other medicines. The long-term benefits would be even larger, perhaps more than a 75 percent overall risk reduction, since risk is only partially reversed in the first one to two years of blood pressure and cholesterol lowering treatment.

Considering the above unmet needs, it would be beneficial to have an effective and convenient therapy and formulations, comprising multiple cardiovascular disease-preventive medicines that would effectively reduce the risk of cardiovascular events. In conventional therapy, patients at higher risk of cardiovascular events frequently are on multiple drug therapy, taking two or more different medications at the same time. Presenting multiple medications in a single fixed dose composition promotes patient compliance by avoiding the inconvenience of taking multiple doses of medicines in a single day, and reducing the chance of skipping doses.

U.S. Pat. No. 6,235,311 discloses a pharmaceutical composition which is useful for cholesterol lowering and reducing the risk of a myocardial infarction, the composition includes a statin, such as pravastatin, lovastatin, simvastatin, atorvastatin, cerivastatin, or fluvastatin, in combination with aspirin, in a manner to minimize chemical interactions between aspirin and the statin, and to minimize the side effects of aspirin.

U.S. Pat. Nos. 6,121,249 and 6,323,188 disclose a method of reducing the incidence and severity of arteriosclerosis, atherosclerotic central nervous system disease, claudication, coronary artery disease, homocystine-related disorders, hypertension, peripheral vascular disease, presenile dementia, and restenosis in humans by daily administration of an effective amount of a combination of acetylsalicylic acid (ASA, or aspirin), at least one antioxidant, a cyanocobalamin compound (Vitamin B₁₂), a folic acid compound, a pyridoxine compound (Vitamin B₆), and a niacin compound.

U.S. Pat. No. 5,622,985 discloses that inhibitors of 3-hydroxy-3-methylglutaryl coenzyme A (HMG CoA) reductase, also called “statins,” particularly pravastatin, when used alone or with an angiotensin converting enzyme (ACE) inhibitor, decrease the risk of a second heart attack in a patient who has a substantially normal cholesterol level.

U.S. Pat. No. 6,576,256 discloses methods and compositions for reducing the risk of cardiovascular events in individuals who are at elevated cardiovascular risk, including individuals who have systemic lupus erythematosus.

The methods comprise administering a combination of a cholesterol-lowering agent, such as an HMG CoA reductase inhibitor, an inhibitor of the renin-angiotensin system, such as an ACE inhibitor, aspirin, and optionally one or more of vitamin B₆, vitamin B₁₂, and folic acid. Pharmaceutical formulations combining all the active agents in unit-dose form for once-daily dosing are also provided.

International Application Publication No. WO 01/15674 relates to a combination of an inhibitor of the renin-angiotensin system, optionally an additional antihypertensive agent, a cholesterol-lowering agent, a diuretic, and aspirin, which can be administered to prevent cardiovascular events.

International Application Publication No. WO 01/76632 discloses a pharmaceutical formulation that contains at least two agents that lower blood pressure, having different modes of action, plus an active agent from at least two of lipid regulating agents; platelet function altering agents; and serum homocysteine lowering agents. It is desired in this document to provide at least some of the drugs in smaller amounts than their customary therapeutic doses.

Indian Patent Application No. 153/MUM/2003 discloses a combination package for secondary prevention of coronary artery disease, comprising one or more of the following combinations of individual drug formulations: a) a statin and aspirin; b) a statin, aspirin, and a beta blocker; c) a statin, aspirin, beta blocker, and ACE inhibitor; and d) a statin, aspirin, beta blocker, and angiotensin II antagonist; in therapeutically effective daily dosages.

European Patent No. 1272220 discloses a method for the prevention of cardiovascular disease, a formulation for the prevention of cardiovascular disease, the use of specified active principals for the manufacture of such a formulation for use in the method and a method of preparing said formulation. The formulation comprises at least two blood pressure lowering agents, each selected from a different physiological mode of action selected from a diuretic, a beta blocker, an angiotensin converting enzyme (ACE) inhibitor, an angiotensin II receptor antagonist, and a calcium channel blocker, and an active principle from at least two of the following three categories: i) at least one lipid-regulating agent; ii) at least one platelet function altering agent; and iii) at least one serum homocysteine lowering agent.

An article by N. J. Wald et al., “A Strategy to Reduce Cardiovascular Disease by More Than 80%,” British Medical Journal, Vol. 326, pp. 1419-1423, 2003, advocates the daily prophylactic treatment of everyone over age 55, and everyone with existing cardiovascular disease, with a “polypill” containing the following six drugs: a drug to lower cholesterol, such as either atorvastatin (10 mg) or simvastatin (40 mg); the combination of three blood pressure lowering drugs from different classes, such as a thiazide, a beta-blocker, and an ACE inhibitor (each at half a usual dose); folic acid (0.8 mg); and aspirin (75 mg).

Earlier approaches to making fixed drug combination (FDC) compositions, particularly comprising one or more antihypertensive drugs and antihyperlipidemic drugs, were primarily in the form of tablets such as bilayer tablets, since it was difficult to make capsule compositions comprising these drugs formulated as separate entities such as mini-tablets or tablets. However such bilayer tablets have very poor storage stability due to interactions between active agents, particularly between a statin and aspirin.

In accordance with the recommendations made by the World Health Organization to develop combination products for cardiovascular therapy and test their efficacy in high risk individuals, it is highly desirable to develop a combination product using a variety of cardiovascular drugs including a beta-adrenergic blocking agent, a diuretic, a cholesterol-lowering agent, an inhibitor of the renin-angiotensin system, aspirin, and optionally an anti-diabetes drug. However, developing stable compositions comprising fixed dose combinations of such drugs such as those comprising at least a statin and aspirin are highly challenging because of tendency for chemical interactions between the drugs of the FDC, thus leading to instability. There still exists a need to provide stable compositions comprising FDCs for efficient management of CVDs.

### SUMMARY

An aspect of the present invention provides compositions comprising fixed dose combinations of active agents, wherein each active agent exhibits desirable bioavailability.

An aspect of the present invention provides fixed dose combinations comprising prophylactic or therapeutic amounts of at least one cholesterol-lowering agent, at least one inhibitor of the renin-angiotensin system, aspirin, and at least one beta-adrenergic receptor blocking agent or diuretic.

An aspect of the present invention provides a stable pharmaceutical composition in the form of a capsule comprising one or more tablets wherein each tablet comprises of one or more active agents selected from cholesterol lowering agent, aspirin, inhibitor of rennin-angiotensin system, beta-adrenergic receptor blocking agent and diuretic; particles or capsule comprising one or more active agents selected from cholesterol lowering agent, aspirin, inhibitor of rennin-angiotensin system, beta-adrenergic receptor blocking agent and diuretic; optionally along with one or more tablets which do not comprise any active agent, wherein the cholesterol lowering agent is separated from aspirin.

An aspect of the present invention provides compositions comprising fixed dose combinations containing at least one cholesterol-lowering agent, at least one inhibitor of the renin-angiotensin system, aspirin, and optionally at least one beta-adrenergic receptor blocking agent or diuretic, together with one or more pharmaceutically acceptable excipients.

In an aspect, the invention provides pharmaceutical dosage forms comprising prophylactic or therapeutic amounts of a cholesterol-lowering agent, an inhibitor of the renin-angiotensin system, aspirin, and at least one beta-adrenergic receptor blocking agent or diuretic, wherein a dosage form is in the form of a capsule.

In an aspect, the invention provides capsule dosage forms comprising compositions of one or more active agents formulated as pellets, granules, mini-tablets, powders, or any mixtures thereof.

In an aspect, the invention provides capsule dosage forms comprising compositions of one or more active agents formulated as microcapsules.

In an aspect, the invention provides stable pharmaceutical compositions in the form of a capsule comprising a tablet comprising a cholesterol-lowering agent as active agent, a tablet comprising aspirin as active agent and a tablet comprising an active agent which is a inhibitor of the renin-angiotensin system and another active agent which is a beta-adrenergic receptor blocking agent or a diuretic, optionally along with one or more pharmaceutical acceptable excipients.

In an aspect, the invention provides pharmaceutical dosage forms in the form of a capsule comprising a tablet comprising simvastatin, a tablet comprising aspirin and a tablet comprising lisinopril and atenolol, wherein one or more of the tablets are optionally coated.

In an aspect, the invention provides pharmaceutical dosage forms in the form of a capsule comprising a tablet comprising simvastatin, a tablet comprising aspirin and a tablet comprising lisinopril and hydrochlorothiazide, wherein one or more of the tablets are optionally coated.

In an aspect, the invention provides pharmaceutical compositions in the form of a capsule comprising a simvastatin tablet, prepared from simvastatin granules having a bulk density between about 0.25 g/mL and about 0.95 g/mL, an aspirin tablet prepared from an aspirin blend having a bulk density between about 0.35 g/mL and about 0.9 g/mL, a tablet comprising lisinopril and atenolol, prepared from a mixture of lisinopril and atenolol granules having bulk density between about 0.25 g/mL and about 0.9 g/mL, or a mixture of lisinopril and hydrochlorothiazide granules having a bulk density between about 0.2 g/mL and about 0.9 g/mL.

In a further aspect, the present invention provides capsule dosage forms, wherein the active agents, prepared in the forms of pellets, granules, mini-tablets, powders, microcapsules, or any mixtures thereof, are filled into capsules, and wherein the capsule sizes range from size 2 to size 00, such as a capsule size 0el.

In a further aspect, the present invention provides capsule dosage forms, wherein the active agents, prepared in the forms of pellets, granules, mini-tablets, powders, microcapsules, or any mixtures thereof, are filled into capsules, and wherein the said capsule have fill weights at least about 100 mg, or at least about 500 mg.

In a further aspect, the present invention provides capsule dosage forms comprising: a) a capsule, tablet, or particles containing aspirin; b) a capsule, tablet, or particles containing simvastatin and lisinopril; and one of hydrochlorothiazide and atenolol; wherein only one of a) and b) is in the form of particles.

In a further aspect, the present invention provides a pharmaceutical composition in the form of capsule that, when tested in in-vitro dissolution apparatus USP Type 1 (Basket), with 900 mL of dissolution medium and 100 rpm rotation, releases not less than about 50% of contained drugs within about 30 minutes.

In an aspect, the present invention also provides processes for preparing stable pharmaceutical compositions comprising: a) preparing a tablet comprising a cholesterol-lowering agent, optionally together with one or more pharmaceutical acceptable excipients; b) preparing a tablet comprising aspirin, optionally together with one or more pharmaceutical acceptable excipients; c) preparing a tablet comprising an inhibitor of the renin-angiotensin system and another active agent, which is a beta-adrenergic receptor blocking agent or a diuretic, optionally together with one or more pharmaceutical acceptable excipients; and d) preparing a capsule comprising the tablets of a), b), and c), optionally together with one or more pharmaceutical acceptable excipients.

In an aspect, the present invention also provides processes for preparing stable and bioavailable compositions comprising fixed dose combinations of active agents, after administration to a healthy human exhibiting peak plasma concentrations of simvastatin in the range of about 2 ng/mL to about 80 ng/mL, and AUC_{(0-∞)} in the range of about 75 ng•hr/mL to about 135 ng•hr/mL.

In an aspect, the present invention also provides processes for preparing stable and bioavailable compositions comprising fixed dose combinations of active agents, after administration to a healthy human exhibiting peak plasma concentrations of aspirin in the range of about 70 ng/mL to about 5400 ng/mL, and AUC_{(0-∞)} in the range of about 100 ng•hour/mL to about 3600 ng•hour/mL.

In an aspect, the present invention also provides processes for preparing stable and bioavailable compositions comprising fixed dose combinations of active agents, after administration to a healthy human exhibiting peak plasma concentrations of lisinopril in the range of about 3 ng/mL to about 195 ng/mL, and AUC_{(0-∞)} in the range of about 40 ng•hour/mL to about 4200 ng•hour/mL.

In an aspect, the present invention also provides processes for preparing stable and bioavailable compositions comprising fixed dose combinations of active agents, after administration to a healthy human exhibiting peak plasma concentrations of atenolol in the range of about 28 ng/mL to about 1360 ng/mL, and AUC_{(0-∞)} in the range of about 128 ng•hour/mL to about 8300 ng•hour/mL.

In an aspect, the present invention also provides processes for preparing stable and bioavailable compositions comprising fixed dose combinations of active agents, after administration to a healthy human exhibiting peak plasma concentrations of hydrochlorothiazide in the range of about 7 ng/mL to about 485 ng/mL, and AUC_{(0-∞)} in the range of about 50 ng•hour/mL to about 3600 ng•hour/mL.

In an aspect, the present invention also provides methods for the treatment or prevention of cardiovascular diseases in a human in need thereof.

In an aspect, the present invention also provides treatment of cardiovascular disease which is one or more of hypertension, dyslipidemia, or clinical evidence of cardiovascular disease.

In an aspect, the present invention further relates to methods of management of subjects having an elevated risk of cardiovascular or cerebrovascular events and other associated diseases or disorders.

In an aspect, the present invention provides methods for using stable pharmaceutical compositions comprising at least one cholesterol-lowering agent, at least one inhibitor of the renin angiotensin system, aspirin, and at least one beta-adrenergic receptor blocking agent or diuretic, for the treatment of patients with cardiovascular disorders, cerebrovascular disease, and other associated disorders.

An aspect of the present invention provides methods for using stable pharmaceutical compositions having any drug-related impurity comprising less than about 5% by weight of the label content of the active agent, and having total drug-related impurities in the composition less than about 7% by weight of the label content of the active agent.

An aspect of the present invention provides stable pharmaceutical compositions containing related impurities of simvastatin less than or equal to about 1% of the content of simvastatin, related impurities of aspirin less than or equal to about 3% of the content of aspirin, related impurities of lisinopril less than or equal to about 1% of the content of lisinopril, related impurities of atenolol less than or equal to about 1% of the content of atenolol, and related impurities of hydrochlorothiazide less than or equal to about 0.2% of the content of hydrochlorothiazide.

### DETAILED DESCRIPTION

In accordance with the present invention, pharmaceutical compositions are provided which comprise a fixed dose combination (FDC) of at least one cholesterol-lowering agent, at least one inhibitor of the renin-angiotensin system, aspirin, and at least one beta-adrenergic receptor blocker or diuretic, which are useful in treatment or reduction of risk of cardiovascular events and associated disorders with minimal or no physico-chemical incompatibility between the active agents. In an aspect, the FDC compositions of the present invention exhibit reduced side effects normally associated with use of such drugs.

In embodiments, the invention provides unit solid dosage forms for oral administration comprising at least four active agents, which include one cholesterol-lowering agent, one inhibitor of the renin-angiotensin system, aspirin, and one beta-adrenergic receptor blocker.

In embodiments, the present invention provides stable pharmaceutical compositions in the form of capsules comprising: a) a tablet comprising a cholesterol-lowering agent; b) a tablet comprising aspirin as an active agent; and c) a tablet comprising an active agent which is an inhibitor of the renin-angiotensin system, and another active agent which is a beta-adrenergic receptor blocking agent or diuretic.

In embodiments, the present invention provides stable pharmaceutical compositions in the form of capsules comprising: a) a tablet comprising a cholesterol-lowering agent; b) a tablet comprising aspirin as an active agent; and c) a tablet comprising an active agent which is an inhibitor of the renin-angiotensin system, and another active agent which is a beta-adrenergic receptor blocking agent or diuretic, wherein the cholesterol lowering agent is separated from aspirin.

In embodiments, the invention provides unit solid dosage forms for oral administration comprising at least four active agents, which include one cholesterol-lowering agent, one inhibitor of the renin-angiotensin system, aspirin, and one diuretic.

The invention also relates to methods for using the FDC compositions for the treatment of patients having an elevated risk of cardiovascular events such as myocardial infarction (heart attack), cardiac arrest, congestive heart failure, stroke, peripheral vascular disease, and claudication. The risk factors associated with such life-threatening events include genetic predisposition, tobacco smoking, diabetes, elevated serum cholesterol, hypertension, systemic lupus erythematosus, prior heart attacks or strokes, hemodialysis, elevated homocysteine levels, obesity, sedentary lifestyles, receiving an organ transplant, and others.

The terms “active agent,” “pharmacologically active agent,” and “drug” are used in the present invention to include the active drug compound as well as any of its pharmaceutically acceptable salts, esters, amides, prodrugs, metabolites, analogs, solvates, hydrates, enantiomers, polymorphs, derivatives, and the like.

The terms “salt” or “pharmaceutically acceptable salt” as used herein refer to salts which are known to be non-toxic and are commonly used in pharmaceutical practice. Such pharmaceutically acceptable salts include metal salts, salts with organic bases, salts with basic amino acids, etc. Metal salts include, for example, alkali metal salts, such as sodium salts and potassium salts, and alkaline earth metal salts, such as calcium, magnesium and barium salts. Salts with organic bases include, for example, salts with trimethylamine, triethylamine, pyridine, picoline, ethanolamine, diethanolamine, triethanolamine, dicyclohexylamine, N,N-dibenzylethylenediamine, etc. Salts with basic amino acids include, for example, salts with arginine, lysine, etc. Acid addition salts such as hydrochloride, sulfate, and succinate salts, and the like, are also included.

The term “acid-labile compound” means any compound, which is not stable in acidic conditions or which undergoes degradation or hydrolysis via acid or proton catalyzed reactions.

The term “therapeutic amount” in connection with a drug indicates the amount of the drug contained in a dose, as customarily prescribed for a primary indication that is within the scope of this invention. These amounts are conveniently summarized for many drugs in the “BNF Recommended Dose” column of tables on pages 11-17 of International Application Publication No. WO 01/76632 (the data in the tables being attributed to the March 2000 British National Formulary), and can also be found in other standard formularies and other drug prescribing directories. For some drugs, the prescribed dose may vary based on the nature and severity of the indication.

The term “prophylactic amount” in connection with a drug indicates the amount of the drug contained in a prescribed dose which is useful in reducing the risk and/or severity of CVDs in a subject.

This invention employs any effective cholesterol-lowering agent or combination of such agents. Useful cholesterol-lowering agents include HMG CoA reductase inhibitors, bile acid sequestrants, probucol, and fibric acid agents. HMG CoA reductase inhibitors are competitive inhibitors of HMG CoA reductase, the rate-limiting step in cholesterol bio-synthesis. They occupy a portion of the binding site of HMG CoA, blocking access of this substrate to the active site on the enzyme. HMG CoA reductase inhibitor drugs include lovastatin, atorvastatin, simvastatin, fluvastatin, rosuvastatin, and pravastatin.

The renin-angiotensin system plays a major role in regulating blood pressure. Renin, an enzyme, functions by acting on angiotensinogen to form the decapeptide angiotensin I. Angiotensin I is rapidly converted to the octapeptide angiotensin II by angiotensin converting enzyme (ACE). Angiotensin II acts by numerous mechanisms to raise blood pressure, including raising total peripheral resistance. Inhibitors of the renin-angiotensin system are classified as ACE inhibitors and angiotensin II receptor antagonists, or angiotensin receptor blockers (ARBs). ACE inhibitor drugs include enalapril, lisinopril, perindopril, trandolapril, and ramipril. Angiotensin receptor blocker drugs include losartan, irbesartan, olmesartan, candesartan, valsartan, and telmisartan.

Cyclooxygenase inhibitors are useful in the present invention, due to their ability to affect platelets; the most widely used and studied cyclooxygenase inhibitor is aspirin, which has been shown to prevent myocardial infarction and strokes due to thrombosis, when administered in low daily doses over a long term to patients at risk for cardiovascular events. When sufficient aspirin is present in the circulatory system, platelets that are being formed have an impaired ability to aggregate over their entire 7-10 day lifetimes.

Diuretics increase the rate of urine flow and sodium excretion, and are in a variety of classes such as inhibitors of carbonic anhydrase, loop diuretics, thiazides and thiazide-like diuretics, potassium sparing diuretics, and antagonists of mineralocorticoid receptors. Thiazides and thiazide-like derivatives include bendroflumethazide, chlorothiazide, hydrochlorothiazide, and hydroflumethazide. Hydrochlorothiazide acts by blocking salt and fluid re-absorption in the kidneys, causing increased urine output (diuresis). It has also been widely used in treating mild hypertension.

Beta-adrenergic receptor antagonists block the action of the sympathetic nervous system and a portion of the involuntary nervous system. By blocking the action of these nerves, they reduce the heart rate and are useful in treating abnormally rapid heart rhythms. These drugs also reduce the force of heart muscle contractions and lower blood pressure. By reducing the heart rate and the force of muscle contraction, beta-blockers reduce heart muscle oxygen demand. Beta-adrenergic blocking agents include atenolol, metoprolol and propranolol.

In accordance with embodiments of the present invention, various dosage forms that can effectively administer the drug combination include tablets, capsules, and caplets, and may also comprise a plurality of granules, beads, powders, or pellets that may or may not be encapsulated. In embodiments, the FDC compositions of the present invention are capsule dosage forms.

In embodiments, the invention provides FDC compositions in capsule dosage form for oral administration, comprising four active agents: simvastatin, in an amount of about 0.1% to about 40% by weight of the composition; lisinopril, in an amount of about 0.1% to about 40% by weight of the composition; aspirin, in an amount of about 1% to about 50% by weight of the composition; and either of atenolol, in an amount of about 0.1% to about 30% by weight of the composition, or hydrochlorothiazide, in an amount of about 0.5% to about 30% by weight of the composition; together with one or more pharmaceutically acceptable excipients in an amount of about 30% to about 95% by weight of the composition.

In embodiments, the invention provides FDC compositions in capsule dosage form for oral administration, comprising four active agents: simvastatin, in an amount of about 0.1% to about 40% by weight of the composition; hydrochlorothiazide, in an amount of about 2% to about 40% by weight of the composition; aspirin, in an amount of about 1% to about 50% by weight of the composition; and atenolol, in an amount of about 0.1% to about 30% by weight of the composition; together with one or more pharmaceutically acceptable excipients in an amount of about 30% to about 95% by weight of the composition.

The term “excipient” or “pharmaceutically acceptable excipient” means a component of a pharmaceutical product that is not an active ingredient, such as a filler, diluent, binder, lubricant, disintegrant, carrier, etc. The excipients that are useful in preparing the pharmaceutical compositions are generally safe, non-toxic and neither biologically nor otherwise undesirable, and are acceptable for human pharmaceutical use as well as for veterinary use. An “excipient” or “pharmaceutically acceptable excipient” as used in the specification includes both one and more than one such excipient.

In embodiments, of the present invention provides stable compositions comprising a FDC, wherein one or more of the active agents of the FDC exhibit a modified release. The modified release may be in the form of delayed release, extended release, sustained release, pulsatile release, prolonged release, or any combinations thereof. “Modified release” refers to systems where the drug release into aqueous fluids does not commence immediately after a dosage form contacts an aqueous environment, and/or where drug release has a rate that is slower than would be expected from the immediate release dosage form.

Combining two or more active ingredients in single dosage form requires consideration of the possibility for chemical interactions between the drug substances. Certain statin compounds are known to be susceptible to degradation and/or oxidation when subjected to unfavorable physical and/or chemical conditions. Acidic active ingredients like aspirin can react with basic drugs, and acidic ingredients such as aspirin can facilitate the degradation of acid labile drugs such as simvastatin.

In embodiments of the invention, a FDC composition comprising four active drug substances can advantageously be prepared as a single solid dosage form in such a manner that any of the one or more drugs that show a tendency for interactions are formulated in separate entities. Thus, the active substances are effectively prevented from any drug-drug interaction; and the stability of the combination drug product can be maximized due to the possibility of optimizing the formulations of each of the active substances with respect to physical and/or chemical interactions.

Stable compositions comprising a FDC in which each of the active agents provide the desired in vitro and in vivo drug release, and bioavailability, can be prepared according to the invention. In an approach, capsules comprise separate mini-tablets of one or more of the active ingredients, such that the drugs that might interact are made into separate mini-tablets. Mini-tablets are prepared of individual components of a combination using pharmaceutical procedures of tablet making such as direct compression, dry granulation, or wet granulation. The individual mini-tablets are filled into hard gelatin capsules. A final dosage form may comprise one or more than one mini-tablet of each individual component. Further, these mini-tablets may be film coated or enteric coated.

In embodiments of the present invention, in order to avoid interactions between drugs, some active ingredients of a said combination can be formulated as mini-tablets and the others filled into the capsules as powders, granules, or beads. The mini-tablets can be optionally film coated or enteric coated. In an aspect, a dosage form in the form of a capsule comprises one or more mini-tablets and a powder, or two or more mini-tablets, or one or more mini-tablets and granules or beads.

In embodiments, the compositions of the present invention provide a fixed dose of simvastatin in the range of about 5-80 mg, lisinopril in the range of about 2.5-20 mg, aspirin in the range of about 25-600 mg, and atenolol in the range of about 25-100 mg.

In embodiments, the compositions of the present invention provide a fixed dose of simvastatin in the range of about 5-80 mg, hydrochlorothiazide in the range of about 10-100 mg, aspirin in the range of about 25-600 mg, and atenolol in the range of about 25-100 mg.

In an embodiment, a unit dose can be administered once-daily, to provide a therapeutic or prophylactic effect.

Useful pharmaceutically acceptable excipients of the present invention include, but are not limited to, diluents, disintegrants, binders, lubricants, antioxidants, surfactants, pH modifiers, antiadherants, and the like, and any combinations thereof. Diluents that can be used in pharmaceutical formulations of the present invention include, but are not limited to, microcrystalline cellulose (“MCC”), silicified MCC (e.g. PROSOLV™ HD 90), microfine cellulose, lactose, starch, pregelatinized starch, sugar, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide, and any mixtures thereof.

Binders can be used in the pharmaceutical compositions of the present invention to help hold tablets together after compression. Some typical binders are acacia, guar gum, alginic acid, carbomers (e.g. Carbopol™ products), dextrin, maltodextrin, methylcelluloses, ethylcelluloses, hydroxyethyl celluloses, hydroxypropyl celluloses (e.g. KLUCEL™), hydroxypropyl methylcelluloses (e.g. METHOCEL™), carboxymethylcellulose sodiums, liquid glucose, magnesium aluminum silicate, polymethacrylates, polyvinylpyrrolidones (e.g., povidone K-90 D, KOLLIDON™), copovidone (PLASDONE™), gelatin, starches, and any mixtures thereof.

Disintegrants that can be used in pharmaceutical formulations of the present invention include, but are not limited to, methylcelluloses, microcrystalline celluloses, carboxymethyl cellulose calcium, carboxymethyl cellulose sodium (e.g. AC-DI-SOL™, PRIMELLOSE™), crospovidones (e.g. KOLLIDON™ POLYPLASDONE™), povidones, guar gum, magnesium aluminum silicate, colloidal silicon dioxide (AEROSIL™), polacrilin potassium, starch, pregelatinized starch, sodium starch glycolate (e.g. EXPLOTAB™), sodium alginate, and any mixtures thereof.

The compositions may include additional pharmaceutically acceptable excipients, including any one or more of glidants, lubricants, surfactants such as sodium lauryl sulphate, and other commonly used excipients. This list, and the foregoing listings of representative specific excipients, is not intended to be exhaustive, as those skilled in the art will be aware of other substances that can be used.

Formulations of the present invention may include antioxidants, including, but not limited to, ascorbic acid and its esters, butylated hydroxytoluene (BHT), butylated hydroxyanisole (BHA), α-tocopherol, cystein, citric acid, propyl gallate, sodium bisulfate, and any mixtures thereof.

In embodiments, the present invention provides stable pharmaceutical compositions in the form of capsules comprising one or more tablets, wherein each tablet comprises one or more active agents, optionally together with one or more excipients, the capsules comprising at least four active agents: a cholesterol lowering agent; aspirin; an inhibitor of the renin-angiotensin system; and a beta-adrenergic receptor blocking agent or diuretic.

In embodiments, the present invention provides processes for making the FDC compositions. The processes for manufacturing a formulation of the present invention are not limited to the processes described herein and the formulations can be prepared by using any of processes known to one skilled in the art. One, or more than one, active ingredient can be used, together with or without directly compressible grade excipients, by granulation together or separately, using wet granulation or dry granulation, with or without other excipients. Further, one or more than one active agents can be granulated while the others may be used without granulation. Spray drying and spray granulation techniques may also be used to prepare compositions of the present invention.

In embodiments, pharmaceutical compositions of the present invention are manufactured as follows: active agents are prepared by sifting at least one active agent and one or more excipients through a desired mesh size sieve and then mixing, using a rapid mixer granulator, planetary mixer, mass mixer, ribbon mixer, fluid bed processor, or any other suitable device. The blend can be granulated, such as by adding a solution or suspension with or without a binder, whether alcoholic or hydro-alcoholic or aqueous, in a low or high shear mixer, fluidized bed granulator and the like, or by dry granulation. The granules can be dried using a tray dryer, fluid bed dryer, rotary cone vacuum dryer, and the like. The granules can be sized using an oscillating granulator or comminuting mill or any other conventional equipment equipped with a suitable screen. Alternatively, granules can be prepared by extrusion and spheronization, or roller compaction. Also, the manufacture of granules containing active agents can include mixing with directly compressible excipients or roller compaction.

The granules obtained by a dry or wet technique can be blended with one or more lubricants and/or anti-adherants. The lubricated blend can be compressed using a suitable device, such as a rotary machine to form slugs, which are passed through a mill or fluid energy mill or ball mill or colloid mill or roller mill or hammer mill and the like, equipped with a suitable screen to obtain the milled slugs of actives.

In other embodiments of the invention, small tablets (mini-tablets) can be made by compressing granules, using dies and punches of various sizes and shapes, as desired. Optionally, a coating can be applied to the tablets, if desired, by techniques known to one skilled in the art such as spray coating, dip coating, fluidized bed coating and the like.

In embodiments of the present invention, suitable solvent systems such as alcoholic, hydroalcoholic, aqueous, or organic may be used to facilitate processing.

In embodiments of the invention, granules obtained by a dry or wet technique can be blended with one or more lubricants and/or anti-adherants and then filled into single capsule or into different capsules of different sizes, such that a smaller capsule can be filled into another larger capsule.

In embodiments of the present invention, granules comprising lisinopril and atenolol are filled into a smaller capsule, and then granules comprising aspirin and simvastatin are filled into a larger capsule together with the smaller capsule.

In accordance with embodiments of the present invention, FDC compositions in capsule dosage form for oral administration comprise four active agents; simvastatin; aspirin; lisinopril; and atenolol or hydrocholorothiazide; in the form of tablets, wherein one or more tablets are coated with a polymer or a sugar.

In embodiments of the present invention FDC compositions in capsule dosage form for oral administration comprise four active agents: simvastatin; aspirin; lisinopril; and atenolol or hydrocholorothiazide; in the form of tablets where aspirin can be in a delayed release tablet.

In embodiments, the present invention provides small tablets (mini-tablets), pellets, granules, and/or powders filed into capsules, wherein the capsule sizes are in the size range of size 1 to size 00, or size 1 to size 0el, for enhancing patient compliance, since the patient needs to consume only a single dosage form. Approximate commercially available hard gelatin capsule body volumes are

| Capsule Size | Volume (mL) |
|---|---|
| 00 | 0.95 |
| 0el | 0.78 |
| 0 | 0.68 |
| 1 | 0.50 |
| 2 | 0.37 |

In embodiments of the present invention, capsule dosage forms have a fill weight between about 100 mg and about 900 mg.

In embodiments, FDC compositions of the present invention comprise mini-tablets filled into capsules optionally together with pharmaceutical acceptable excipients. Mini-tablets can be filled into capsules using an automated capsule filling machine. The mini-tablets can have any desired sizes and shapes.

In embodiments, the tablets are small (mini-tablets). Mini-tablets frequently have diameters about 1 mm to about 5 mm, or less. More than one of the mini-tablets can be loaded into a single capsule to provide a single fixed unit dose.

In embodiments, the present invention provides pharmaceutical capsule dosage forms comprising therapeutic amounts of simvastatin, present in granules; and lisinopril, aspirin, and either or both of atenolol or hydrochlorothiazide present in mini-tablets, wherein bulk densities of the granules comprising simvastatin are between about 0.25 g/mL and about 0.95 g/mL.

In embodiments, the present invention provides pharmaceutical capsule dosage forms comprising therapeutic amounts of aspirin, present in granules, and simvastatin, lisinopril, and either or both of atenolol and hydrochlorothiazide, present in mini-tablets, wherein bulk densities of granules comprising aspirin are between about 0.35 g/mL and about 0.9 g/mL.

In embodiments, the present invention provides pharmaceutical capsule dosage forms comprising therapeutic amounts of lisinopril and hydrochlorothiazide, present in granules, and simvastatin and aspirin, present in mini-tablets, wherein the bulk densities of granules comprising lisinopril and hydrochlorothiazide are between about 0.2 g/mL and about 0.95 g/mL.

In embodiments, the present invention provides pharmaceutical capsule dosage forms comprising therapeutic amounts of lisinopril and atenolol, present in granules, and simvastatin and aspirin, present in mini-tablets, wherein the bulk densities of granules comprising lisinopril and atenolol are between about 0.25 g/mL and about 0.9 g/mL.

Since the active agents used in the FDC compositions are susceptible to certain types of degradation/oxidation/drug-drug interactions, there is a possibility of formation of several undesirable impurities during the manufacturing process and/or storage of the formulation. It is therefore important to minimize the formation of such undesirable impurities in the composition. The FDC compositions of the present invention are found to be sufficiently stable and show minimized decomposition or impurity formation, and even if an impurity is formed, is it is present within acceptable and prescribed limits.

In embodiments of the present invention, the impurities formed in the FDC compositions of the present invention are within acceptable limits, e.g., for an active agent of the FDC the individual known impurities do not exceed more than about 0.5% of the label content of the active agent, during the course of storage, according to the International Conference on Harmonization (ICH) guidelines for low dose drugs.

In embodiments, the present invention provides stable pharmaceutical compositions comprising a FDC, wherein any individual impurity chemically related to an active agent in the composition is less than about 5%, and the total impurities of an active agent in the composition are less than about 7%, based on the label content of the active agent.

In embodiment of the present invention, total impurities chemically related to an active agent of the FDC compositions, formed during processing or storage, are less than about 3% of the label active agent content in the composition.

In embodiment of the present invention, any impurities from an active agent of the FDC compositions, formed during processing or storage, are present in amounts less than about 2% of the label active agent content in the composition.

In embodiments, pharmaceutical compositions comprising a FDC comprise less than about 0.9% of the compound O-monoacetyl atenolol, based on the label atenolol content in the composition.

In embodiments, pharmaceutical compositions comprising a FDC comprise less than about 1% of the compound N-monoacetyl atenolol, based on the label atenolol content in the composition.

In embodiments, pharmaceutical compositions comprising a FDC comprise less than about 1.5% of the compound diacetyl atenolol, based on the label atenolol content in the composition.

In embodiments, pharmaceutical compositions comprising a FDC comprise less than about 0.8% of the compound monoacetyl lisinopril, based on the label lisinopril content in the composition.

The present invention provides FDC compositions, wherein the active agents of the FDC remain stable during manufacturing and during storage for commercially relevant times (e.g., about 6 months, 1 year, 2 years, etc., including any intermediate times), and wherein the active agents provide the desired bioavailability for providing the desired pharmacological action. In an aspect, the present invention provides FDC compositions wherein one active agent may potentiate the pharmacological response of another active agent by improving the pharmacokinetic profile of the other drug. In an aspect, the FDC compositions of the present invention may comprise lower doses of one or more active agent than generally used in the art to achieve the desired pharmacological response.

The present invention provides FDC compositions for which the following related impurities can be present:

Simvastatin-Related Impurities

| Impurity | Structure |
|---|---|
| Hydroxy acid: (3R,5R)-7-[(1S,2S,6R.8S,8a R)- 8-[(2,2-dimethylbulanoyl)oxy]-2,6-dimethyl 1,2,6,7,8,8a-hexahydronaphthalen-1-yl 1]-3,5- dihydroxyheptanoic acid | 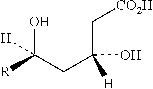 |
| Acetate ester: (1S,3R,7S,8S,8aR)-8-[2- [(2R,4R)-4-(acetyloxy)-6-oxotetrahydro-2H- pyran.2-yl] ethyl]-3,7-dimethyl-1,2,3,7,8,8a- hexahydronaphthalen-1-yl2,2- dimethylbutanoate | 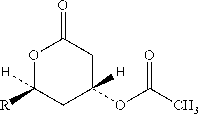 |
| Dehydro simvastatin: (1S,3R,7S,8S,8aR)-3,7- dimethyl-8-[2-[(2R)-6-oxo-3,6-dihydro-2H- pyran-2-yl]ethyl)-1,2,3,7,8,8a- hexahydronaphthalen-1-yl 2,2- dimethylbutanoate | 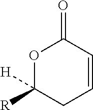 |
| Simvastatin dimer: (2R,4R)-2- [[(1S,25,6R8S,8aR)-8-[(2,2- dimethylbutanoyl)oxy]-2,6-dimethyl- 1,2,6,7,8,8a-nexahydronaphthalen-1-yl]ethyl]- 6-oxotetrahydro-2H-pyran-4-yl (3R,5R)-7- [{1S,2S,6R,8S,8aR)-8-[(2,2- dimethylbutanoyl)oxy]-2,6-dimelhyl- 1,2,6,7,8,8a-hexahydronaphthalen-1-yl]-3,5- dihydroxyhepatanoate | 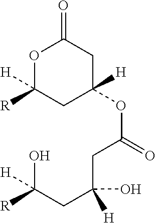 |
| Lovastatin; (1S, 3R,7S,8S,8aR)-8-[2- [(2R,4R)-4-hydroxy-6-oxotetrahydro-2H-pyran- 2-yl[ethyl]-3,7-dimethyl-.1,2,3,7,8,8a- hexahydronaphthalen-1-yl (2S)-2- methylbutanoate | 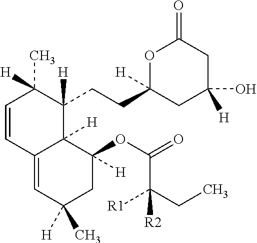 |

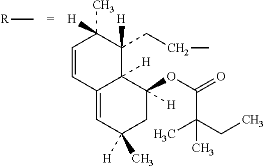

Lisinopril-Related Impurities

| Impurity | Structure |
|---|---|
| CPP lysine: N2-[(1S)-carboxy-3-phenylpropyl]- L-lysine | 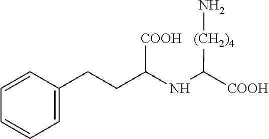 |
| APBA: (2RS)-2-amino-4-phenylbutanoic acid | 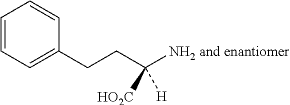 |
| RSS -isomer: (2S)-1-[(2S)-6-amino-2-[[(1R)-1- carboxy-3-phenylpropyl]amino]hexanoyl] pyrrole-2-carboxylic acid | 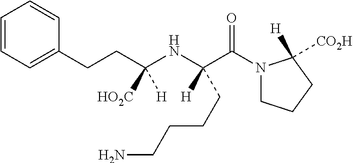 |
| DKP (S,S,S-diketopiperazine): (2S)-2- [(3S,8aS)-3-(4-aminobutyl)-1,4- dioxohexahydropyrrolo[1,2-a]pyrazin-2(1H)- yl]-4-phenylbutanoic acid | 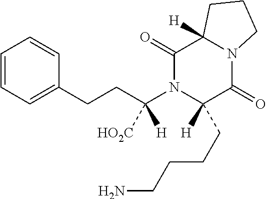 |
| Monoacetyl: 1-[6-acetylamino-2-(1-carboxy-3- phenyl-propylamino)-hexanoyl]-pyrrolidine-2- carboxylic acid | 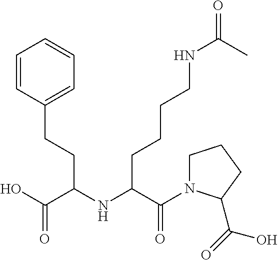 |

Atenolol-Related Impurities

| Impurity | Structure |
|---|---|
| PHPA: 2-(4-hydroxyphenyl)acetamide | 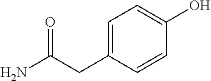 |
| Diol: 2-[4-[(2RS)-2,3- dihydroxypropoxy]phenyl]acetamide | 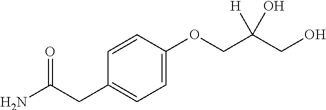 |
| Blocker acid: 2-[4-[(2 RS)-2-hydroxy-3-[(1- methylethyl)amino]propoxy]phenyl]acetic acid | 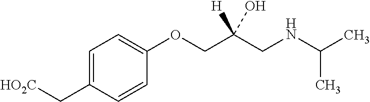 |
| Tertiary amine: 2,2′-[(1- methylethyl)iminobis(2-hydroxypropan-3,1- diyloxy-4,1-phenylene)]diacetamide | 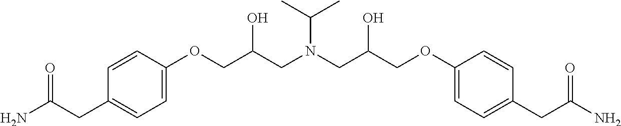 |
| O-mono: 2-(4-Carbamoylmethyl-phenoxyl)-1- (isopropyl amino methyl)-ethyl acetate. | 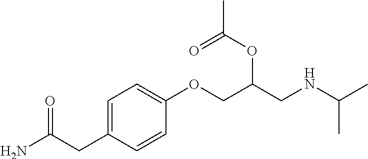 |
| N-MONO: 2-{4-(3-(Acetyl-isopropyl-amino)-2- Hydroxy-propoxy]-phenyl}-acetamide | 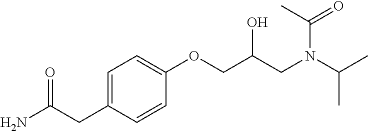 |
| Diacetyl: Acetic acid·1-[(acetyl-isopropyl- amino)-melhyl]-2-(4-carbamoyl methyl- phenoxy)-ethyl ester | 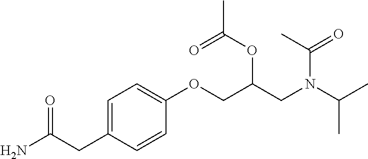 |

Hydrochlorothiazide-Related Impurities

| Impurity | Structure |
|---|---|
| CTZ: chlorothiazide | 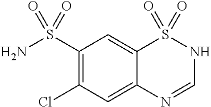 |
| DSA: 6-chloro-N-[(6-chloro-7, sulphamoyl-2,3-dihydro-4H-1,2,4- benzothiadiazin-4-yl1,1- dioxide) methyl]-3,4-dihydro-2H- 1,2,4-benzothiadiazine-7- sulphonamide 1,1-dioxide. | 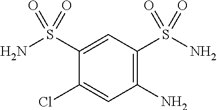 |

Aspirin-Related Impurity

| | |
|---|---|
| Salicylic acid: 2-hydroxybenzoic acid | 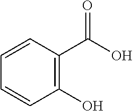 |

In embodiments, the present invent provides pharmaceutical compositions comprising a FDC, wherein: one or more related impurities of simvastatin, such as hydroxyl acid, lovastatin, acetate ester, dehydro simvastatin, and simvastatin dimer, comprise not more than about 1% of the label content of simvastatin; a related impurity of aspirin, which is salicylic acid, comprises not more than about 3% of the label content of aspirin; one or more related impurities of lisinopril, such as mono-acetyl lisinopril, RSS isomer of lisinopril, and SSS DPK, are not more than about 1% of the label content of lisinopril; one or more related impurities of atenolol, such as o-monoacetyl atenolol, N-monoacetyl atenolol, diacetyl atenolol, and diol are not more than about 1% of the label content of atenolol; and one or more of the related impurities of hydrochlorothiazide, such as chlorothiazide and DSA, are not more than about 0.2% of the label content of hydrochlorothiazide.

It is a challenging task to design compositions wherein the desired is pharmacokinetic profiles of each drug are preserved. Usually, in practice, the absorption of one of the active agents may decrease while that of another one increases. When choosing the pharmaceutical excipients, disintegrants, and other auxiliary agents to be used in a pharmaceutical composition in combination with several active agents, numerous factors have to be considered, e.g., the chemical and physical characteristics of the active agents and the auxiliary agents, the bioavailabilities of the active agents, the method of preparing the composition, the stability of the composition, etc. The present invention enables achievement of suitable bioavailability parameters for each active agent, compared to the reference individual formulations of each drug.

Similarly, it is very difficult to achieve comparative in vitro dissolution profiles for different active agents from fixed dose combinations. Usually, in practice, the dissolution of one of the active agents may affect another drug. When choosing the pharmaceutical excipients, disintegrants, and other auxiliary agents to be used in a pharmaceutical composition in combination with several active agents, numerous factors have to be considered, e.g., the chemical and physical characteristics of the active agents and the auxiliary agents, different pH values (5.5-7.5) for testing in physiologic fluids, different ionic strengths of dissolution media, presence of surfactants, varying mechanical stress (speed), buffer composition, dissolution media, etc. Comparative dissolution profiles for each test product herein compare favorably with reference products when individual drug formulations are compared with reference formulations. The dissolution profiles and rates achieved for individual drugs in the FDC are similar to those achieved with single-drug formulations.

In vitro dissolution profiles for pharmaceutical dosage forms can be obtained using methods such as Test 711 “Dissolution” in United States Pharmacopeia 29, United States Pharmacopeia Convention, Rockville, Md., 2005 (“USP”). Useful physiologic dissolution media are described in monographs for individual drug products, and the drug content in a dissolution medium can be determined using techniques such as high performance liquid chromatography.

In embodiments, the present invention provides pharmaceutical compositions comprising a therapeutically effective amount of a fixed dose combination containing simvastatin, aspirin, lisinopril, and either of atenolol or hydrochlorothiazide, as active agents, and pharmaceutically acceptable excipients, wherein each active agent has a dissolution of not less than about 50% of the contained active agent within 30 minutes after immersion in an aqueous medium, using the USP Type 1 (Basket) apparatus, 900 mL dissolution medium, and 100 rpm rotation.

In embodiments, the present invention provides pharmaceutical compositions comprising a therapeutically effective amount of a fixed dose combination containing simvastatin, aspirin, lisinopril, and one of atenolol or hydrochlorothiazide, as active agents, and pharmaceutically acceptable excipients, wherein each active agent exhibits bioequivalence to a reference product of the active agent.

In embodiments, the present invention provides pharmaceutical compositions comprising a therapeutically effective amount of a fixed dose combination containing simvastatin, aspirin, lisinopril, and one of atenolol or hydrochlorothiazide, as active agents, and pharmaceutically acceptable excipients, wherein a composition, after oral administration to healthy humans, provides peak plasma concentrations (Cₘₐₓ) of simvastatin in the range of about 2 ng/mL to about 80 ng/mL, and AUC_{(0-∞)} (area under the plasma concentration-time curve) values in the range of about 75 ng•hour/mL to about 135 ng•hour/mL.

In embodiments, the present invention provides pharmaceutical compositions comprising a therapeutically effective amount of a fixed dose combination containing simvastatin, aspirin, lisinopril, and one of atenolol or hydrochlorothiazide, as active agents, and pharmaceutically acceptable excipients, wherein a composition, after oral administration to healthy humans, to provides peak plasma concentrations (Cₘₐₓ) of aspirin in the range of about 70 ng/mL to about 5400 ng/mL, and AUC_{(0-α)} values in the range of about 100 ng•hour/mL to about 3600 ng•hour/mL.

In embodiments, the present invention provides pharmaceutical compositions comprising a therapeutically effective amount of a fixed dose combination containing simvastatin, aspirin, lisinopril, and one of atenolol or hydrochlorothiazide, as active agents, and pharmaceutically acceptable excipients, wherein a composition, after oral administration to healthy humans, provides peak plasma concentrations (Cₘₐₓ) of lisinopril in the range of about 3 ng/mL to about 195 ng/mL, and AUC_{(0-α)} values in the range of about 40 ng•hour/mL to about 4200 ng•hour/mL.

In embodiments, the present invention provides pharmaceutical compositions comprising a therapeutically effective amount of a fixed dose combination containing simvastatin, aspirin, lisinopril, and atenolol, as active agents, and pharmaceutically acceptable excipients, wherein a composition, after oral administration to healthy humans, provides peak plasma concentrations (Cₘₐₓ) of atenolol in the range of about 28 ng/mL to 1360 ng/mL, and AUC_{(0-α)} values in the range of about 128 ng•hour/mL to about 8300 ng•hour/mL.

In embodiments, the present invention provides pharmaceutical compositions comprising a therapeutically effective amount of a fixed dose combination containing simvastatin, aspirin, lisinopril, and hydrochlorothiazide, as active agents, and pharmaceutically acceptable excipients, wherein the composition, after oral administration to a healthy humans, provides peak plasma concentrations (Cₘₐₓ) of hydrochlorothiazide in the range of about 7 ng/mL to 485 ng/mL, and AUC_{(0-α)} values in the range of about 50 ng•hour/mL to about 3600 ng•hour/m L.

An aspect of the present invention further relates to methods of management of subjects having an elevated risk of cardiovascular events and other associated diseases or disorders.

In an aspect, the present invention provides methods of using a stable pharmaceutical composition comprising at least one cholesterol-lowering agent, at least one inhibitor of the renin-angiotensin system, aspirin, and at least one beta-adrenergic receptor blocking agent or diuretic, for the treatment of patients with cardiovascular disorders and other associated disorders.

The following examples will further describe certain specific aspects and embodiments of the invention in greater detail. These examples are provided only for purposes of illustration and should not be construed as limiting the scope of the invention in any manner.

### EXAMPLE 1

Inner capsule

| Ingredient | mg/Capsule |
|---|---|
| Lisinopril | 5 |
| Atenolol | 25 |
| Dibasic calcium phosphate anhydrous (A-Tab ®) | 87.5 |
| Microcrystalline cellulose (AVICEL ® PH112) | 40 |
| Mannitol (PEARLITOL ® SD 200) | 25 |
| Coloring agent (Spectracol Ponceau ® 4RLK | 0.5 |
| 815633) | |
| Polyvinylpyrrolidone (povidone K-30) | 4 |
| Zinc stearate | 2 |
| Sodium starch glycolate | 8 |
| Zinc stearate | 4 |
| Total weight | 200 |

Manufacturing Process:

1. The first eight ingredients are passed through an ASTM #40 mesh sieve and mixed uniformly.

2. The ingredients of step 1 are dry granulated by roll compaction.

3. The granules are blended with the last two ingredients.

4. The lubricated granules are filled into small capsules.

Outer capsule

| Ingredient | mg/Capsule |
|---|---|
| Simvastatin (with 0.01% butylated | 10 |
| hydroxyanisole) | |
| Acetylsalicylic acid | 75 |
| Lactose monohydrate (Flowlac ® 100) | 68.24 |
| Pregelatinized starch (Starch 1500) | 3.75 |
| Ascorbic acid | 2.5 |
| Butylated hydroxyanisole (BHA) | 0.01 |
| Citric acid anhydrous | 1.25 |
| Microcrystalline cellulose (Avicel PH112) | 2.5 |
| Zinc stearate | 0.5 |
| Pregelatinized starch (Starch 1500) | 6.25 |
| Microcrystalline cellulose (Avicel PH112) | 2.5 |
| Zinc stearate | 2.5 |
| Total weight | 175 |

Manufacturing Process:

1. The first nine ingredients are passed through an ASTM #40 mesh sieve and mixed uniformly.

2. The ingredients of step 1 are dry granulated by roller compaction.

3. The granules are blended with the last three ingredients.

4. A small capsule containing lisinopril and atenolol is placed inside the body of a size 0el outer capsule.

5. Lubricated granules of step 3 are filled into the outer capsule and the capsule is sealed.

### EXAMPLE 2

Inner capsule

| Ingredient | mg/Capsule |
|---|---|
| Lisinopril | 5 |
| Hydrochlorothiazide | 12.5 |
| Dibasic calcium phosphate anhydrous (A-Tab) | 88 |
| Microcrystalline cellulose (Avicel PH112) | 52 |
| Mannitol (Pearlitol SD 200) | 25 |
| Coloring agent (Spectracol Ponceau 4RLK | 0.5 |
| 815633) | |
| Polyvinylpyrrolidone (povidone K-30) | 4 |
| Zinc stearate | 2 |
| Sodium starch glycolate | 8 |
| Zinc stearate | 3 |
| Total weight | 200 |

Manufacturing Process:

1. The first eight ingredients are passed through an ASTM #40 mesh sieve and mixed uniformly

2. The ingredients of step 1 are dry granulated by roller compaction.

3. The granules are blended with the last two ingredients.

4. The lubricated granules are filled into small capsules.

Outer capsule

| Ingredient | mg/Capsule |
|---|---|
| Simvastatin (with 0.01% BHA) | 10 |
| Acetylsalicylic acid | 75 |
| Lactose monohydrate (Flowlac 100) | 68.24 |
| Pregelatinized starch (Starch 1500) | 3.75 |
| Ascorbic acid | 2.5 |
| Butylated hydroxyanisole | 0.01 |
| Citric acid anhydrous | 1.25 |
| Microcrystalline cellulose (Avicel PH112) | 2.5 |
| Zinc stearate | 0.5 |
| Pregelatinized starch (Starch 1500) | 6.25 |
| Microcrystalline cellulose (Avicel PH112) | 2.5 |
| Zinc stearate | 2.5 |
| Total weight | 175 |

Manufacturing Process:

1. The first nine ingredients are passed through an ASTM #40 mesh sieve and mixed uniformly.

2. The ingredients of step 1 are dry granulated by roller compaction.

3. The granules are blended with the last two ingredients.

4. A small capsule containing lisinopril and atenolol is placed inside the body of a size 0el outer capsule.

5. Lubricated granules of step 3 are filled into the outer capsule and the capsule is sealed.

### EXAMPLE 3

Simvastatin tablet

| Ingredient | mg/Tablet |
|---|---|
| Simvastatin (with 0.01% BHA) | 40 |
| Lactose monohydrate | 212.71 |
| Microcrystalline cellulose (Avicel | 8.5 |
| PH101) | |
| Pregelatinized starch (Starch 1500 | 21 |
| LM) | |
| Ascorbic acid | 10 |
| Butylated hydroxyanisole | 0.04 |
| Citric acid anhydrous | 5 |
| Isopropyl alcohol* | q.s. |
| Water* | q.s. |
| Pregelatinized starch (Starch 1500 | 31.25 |
| LM) | |
| Microcrystalline cellulose (Avicel | 8.5 |
| PH101) | |
| Magnesium stearate | 3 |
| Total weight | 340 |

*Evaporates during processing.

Manufacturing Process:

1. Intragranular ingredients simvastatin, lactose, microcrystalline cellulose (first quantity), starch, citric acid, and ascorbic acid are sifted through an ASTM #30 mesh sieve and mixed in a rapid mixer granulator (RMG) for 15 minutes.

2. Binder solution is prepared by dissolving BHA in isopropyl alcohol and this is added to water with continuous stirring. The dry mixture of 1 is granulated in the RMG with the binder solution.

3. The granules are dried in a fluid bed dryer (FBD) at about 50±5° C. until the loss on drying (LOD) at 105° C. is 1.5-2.5%.

4. Dried granules are passed through an ASTM #30 mesh sieve and the retained particles are milled through a comminuting mill, using a 1.5 mm screen at slow speed, so that all of the granules pass through the sieve.

5. Extragranular microcrystalline cellulose (second quantity) and pregelatinized starch are sifted through an ASTM #30 mesh sieve and blended with the granules in a double cone blender for 10 minutes.

6. The material of 5 is blended with magnesium stearate (previously sifted through an ASTM #60 mesh sieve) in a double cone blender for 5 minutes.

7. The blend of 6 is compressed into tablets with 11.4×5.5 mm caplet-shaped tooling.

Aspirin tablet

| Ingredient | mg/Tablet |
|---|---|
| Aspirin | 75 |
| Lactose monohydrate (Flowlac 100) | 50 |
| Pregelatinized starch (Starch 1500 LM) | 4.675 |
| Opadry AMB Translucent (OY-B-29000) | 3.9 |
| Film coating | |
| Isopropyl alcohol* | q.s. |
| Water* | q.s. |
| Total weight | 133.9 |

*Evaporates during processing.

Manufacturing Process:

1. Lactose monohydrate and pregelatinized starch are sifted through an ASTM #30 mesh sieve and aspirin is sifted through an ASTM #20 mesh sieve.

2. Sifted materials are blended in a double cone blender for 15 minutes.

3. Blend of 2 is compressed into tablets using 6.2 mm round tooling.

4. The tablets are film coated in Ganscota coating equipment using Opadry AMB Translucent (OY-B-29000) in a mixture of water and isopropyl alcohol, and dried.

Lisinopril + atenolol tablet

| Ingredient | mg/Tablet |
|---|---|
| Lisinopril | 10 |
| Atenolol | 50 |
| Microcrystalline cellulose (Avicel PH200) | 19.34 |
| Sodium starch glycolate | 18 |
| Iron oxide red | 0.15 |
| Povidone (PVP K-30) | 2 |
| Isopropyl alcohol* | q.s. |
| Microcrystalline cellulose (Avicel PH200) | 13.51 |
| Sodium starch glycolate | 14 |
| Magnesium stearate | 3 |
| Opadry AMB Translucent (OY-B-29000) | 3.9 |
| Isopropyl alcohol* | q.s. |
| Water* | q.s. |
| Total weight | 133.9 |

*Evaporates during processing.

Manufacturing Process:

1. Intragranular ingredients lisinopril, atenolol, microcrystalline cellulose (first quantity), sodium starch glycolate (first quantity), and iron oxide red are sifted through an ASTM #30 mesh sieve, then are mixed in a RMG for 15 minutes.

2. Binder solution is prepared by dissolving povidone in isopropyl alcohol (first quantity), then the dry mixture is granulated in the RMG using the binder solution.

3. The granules are dried in a FBD at 40±5° C. until the LOD is 1-2%.

4. The dried granules are passed through an ASTM #30 mesh sieve and the retained particles are milled through a comminuting mill, using a 1.5 mm screen, until all granules pass through the sieve.

5. Extragranular microcrystalline cellulose (second quantity) and sodium starch glycolate (second quantity) are sifted through an ASTM #30 mesh sieve, added to the granules, and blended in a double cone blender for 10 minutes.

6. Magnesium stearate is sifted through an ASTM #60 mesh sieve, added to the double cone blender and blended for 5 minutes.

7. The blend of 6 is compressed into tablets using 6.2 mm round shaped tooling.

8. Compressed tablets are film coated in Ganscota coating equipment using Opadry AMB Translucent (OY-B-29000) in a mixture of water and isopropyl alcohol (second quantity), and dried.

Capsule Filling

One of each tablet prepared above is filled into a size 0el capsule.

Dissolution profiles for drugs in capsules prepared above are determined in various media using the USP procedure and the following conditions: Type 1 (Basket) apparatus; 900 mL medium; and 100 rpm rotation. Values in the following tables are cumulative percentages of the specified drug that dissolves in the specified times.

Simvastatin

| Medium | 30 Minutes | 60 Minutes |
|---|---|---|
| pH 2.1 SGF (simulated gastric | 93 | 96 |
| fluid, USP) | | |
| pH 4.5 USP + 0.5% sodium | 96 | 96 |
| lauryl sulfate (SLS) | | |
| pH 6.5 SIF (simulated intestinal | 9 | 10 |
| fluid, USP) | | |

Lisinopril

| Medium | 30 Minutes | 60 Minutes |
|---|---|---|
| pH 4.5 Acetate buffer USP | 97 | 96 |
| 0.1N HCl | 97 | 96 |
| pH 2.1 SGF | 102 | 105 |
| pH 6.8 Phosphate buffer USP | 96 | 96 |
| pH 6.5 SIF | 92 | 97 |

Aspirin

| Medium | 30 Minutes | 60 Minutes |
|---|---|---|
| 0.01N HCl | 87 | 89 |
| 0.1N HCl | 96 | 98 |
| pH 2.1 SGF | 99 | 100 |
| pH 4.5 Acetate buffer USP | 88 | 89 |

Atenolol

| Medium | 30 Minutes | 60 Minutes |
|---|---|---|
| pH 4.5 Acetate buffer USP | 96 | 98 |
| 0.1N HCl | 101 | 102 |
| pH 2.1 SGF | 98 | 98 |
| pH 6.8 Phosphate buffer USP | 98 | 98 |
| pH 6.5 SIF | 98 | 99 |

Capsules prepared above are stored in closed high-density polyethylene (HDPE) bottles at 40° C. and 75% relative humidity for 6 months, and are analyzed for drug content and the levels of drug-related impurity formation. The results are shown below, where the amounts are percentages of the label content of the related active ingredient.

Assay

| Drug | Initial | 6 Months |
|---|---|---|
| Lisinopril | 98.5 | 98.7 |
| Atenolol | 100.3 | 98.7 |
| Simvastatin | 102.8 | 100.3 |
| Aspirin | 100.3 | 101.5 |

Impurities

| Drug | Impurity | Initial | 6 Months |
|---|---|---|---|
| Lisinopril | Monoacetyl | ND | ND |
| | SSS DKP | ND | 0.009 |
| | RSS isomer | 0.09 | 0.12 |
| Atenolol | O-Monoacetyl | ND | ND |
| | N-Monoacetyl | 0.001 | 0.002 |
| | Diacetyl | 0.004 | 0.007 |
| | DIOL | 0.108 | 0.108 |
| | PHPA | 0.022 | 0.047 |
| | Tertiary amine | 0.028 | 0.029 |
| | Blocker acid | 0.049 | 0.04 |
| Simvastatin | Hydroxy acid | 0.35 | 0.19 |
| | Lovastatin | 0.39 | 0.40 |
| | Acetate ester | ND | ND |
| | Dehydro | 0.07 | 0.13 |
| | Dimer | 0.13 | 0.12 |
| Aspirin | Salicylic acid | 0.17 | 0.68 |

ND = Below the limit of detection.

The formulation is considered to have commercially acceptable storage stability.

### EXAMPLE 4

Simvastatin tablet

| Ingredient | mg/Tablet |
|---|---|
| Simvastatin (with 0.01% BHA) | 40 |
| Lactose monohydrate | 212.71 |
| Microcrystalline cellulose (Avicel PH101) | 8.5 |
| Pregelatinized starch (Starch 1500 LM) | 21 |
| Ascorbic acid | 10 |
| Butylated hydroxyanisole | 0.04 |
| Anhydrous citric acid | 5 |
| Isopropyl alcohol* | q.s. |
| Water* | q.s. |
| Pregelatinized starch (Starch 1500 LM) | 31.25 |
| Microcrystalline cellulose (Avicel PH101) | 8.5 |
| Magnesium stearate | 3 |
| Total weight | 340 |

*Evaporates during processing.

Manufacturing Process:

1. Intragranular ingredients simvastatin, lactose, microcrystalline cellulose (first quantity), starch, citric acid and ascorbic acid are sifted through an ASTM #30 mesh sieve and mixed in a rapid mixer granulator (RMG) for 15 minutes.

2. Binder solution is prepared by dissolving BHA in isopropyl alcohol and this is added to water under continuous stirring. The dry mixture of 1 is granulated in the RMG with the binder solution.

3. The granules are dried in a fluid bed dryer (FBD) at about 50±5° C. until the LOD at 105° C. is 1.5-2.5%.

4. Dried granules are passed through an ASTM #30 mesh sieve and the retained particles are milled through a comminuting mill, using a 1.5 mm screen at slow speed, so that all of the granules pass through the sieve.

5. Extragranular microcrystalline cellulose (second quantity) and pregelatinized starch (second quantity) are sifted through an ASTM #30 mesh sieve and blended with the granules in a double cone blender for 10 minutes.

6. The material of 5 is blended with magnesium stearate (previously sifted through an ASTM #60 mesh sieve) in a double cone blender for 5 minutes.

7. The blend of 6 is compressed into tablets using 11.4×5.5 mm caplet-shaped tooling.

Aspirin tablet

| Ingredient | mg/Tablet |
|---|---|
| Aspirin | 75 |
| Lactose monohydrate (Flowlac 100) | 50 |
| Pregelatinized starch (Starch 1500 LM) | 4.675 |
| Opadry AMB Translucent (OY-B-29000) | 3.9 |
| Isopropyl alcohol* | q.s. |
| Water* | q.s. |
| Total weight | 133.9 |

*Evaporates during processing.

Manufacturing Process:

1. Lactose monohydrate and pregelatinized starch are sifted through an ASTM #30 mesh sieve and aspirin is sifted through an ASTM #20 mesh sieve.

2. Sifted materials are blended in a double cone blender for 15 minutes.

3. The blend of 2 is compressed into tablets using 6.2 mm round tooling.

4. The tablets are film coated in Ganscota coating equipment using Opadry AMB Translucent (OY-B-29000) in a mixture of water and isopropyl alcohol, and dried.

Lisinopril + hydrochlorothiazide tablet

| Ingredient | mg/Tablet |
|---|---|
| Lisinopril | 10 |
| Hydrochlorothiazide | 12.5 |
| Dibasic calcium phosphate anhydrous (A-Tab) | 47.33 |
| Microcrystalline cellulose (Avicel PH112) | 25.44 |
| Mannitol | 14.63 |
| Iron oxide red | 0.15 |
| Polyvinylpyrrolidone (PVP K-30) | 2.34 |
| Zinc stearate | 1.17 |
| Sodium starch glycolate | 4.68 |
| Zinc stearate | 1.76 |
| Total weight | 120 |

Manufacturing Process:

1. The first six ingredients are sifted through an ASTM #30 mesh sieve, mixed in a double cone blender for 10 minutes and sifted through an ASTM #30 mesh sieve. Iron oxide red and zinc stearate (first quantity) are sifted through an ASTM #40 mesh sieve, added to the blender, and mixed for 15 minutes.

2. The mixture is compacted into slugs using a roller compactor.

3. The slugs are milled through a comminuting mill, fitted with a 4 mm screen, and passed through an ASTM #18 mesh sieve. Retained particles are milled through a comminuting mill, fitted with a 2.5 mm screen, until all particles pass through an ASTM #18 mesh sieve.

4. The particles are blended in a double cone blender for 10 minutes, then zinc stearate (second quantity) and sodium starch glycolate are added to the blender and blended for 5 minutes.

5. The blend of 4 is compressed into tablets using 6.2 mm round-shaped tooling.

Capsule Filling

One of each tablet prepared above is filled into a size 0el capsule.

Dissolution profiles for the prepared capsules are determined in various media using the USP procedure and the following conditions: Type 1 (Basket) apparatus; 900 mL medium; and 100 rpm rotation. Values in the following tables are cumulative percentages of the specified drug that dissolves in the specified times.

Simvastatin

| Medium | 30 Minutes | 60 Minutes |
|---|---|---|
| pH 2.1 SGF | 93 | 96 |
| pH 4.5 USP + 0.5% SLS | 96 | 96 |
| pH 6.5 SIF | 9 | 10 |

Lisinopril

| Medium | 30 Minutes | 60 Minutes |
|---|---|---|
| pH 4.5 Acetate buffer USP | 100 | 102 |
| 0.1N HCl | 100 | 100 |
| pH 2.1 SGF | 95 | 98 |
| pH 6.8 Phosphate buffer USP | 94 | 95 |

Aspirin

| Medium | 30 Minutes | 60 Minutes |
|---|---|---|
| 0.01N HCl | 87 | 89 |
| 0.1N HCl | 96 | 98 |
| pH 2.1 SGF | 99 | 100 |
| pH 4.5 Acetate buffer USP | 88 | 89 |

Hydrochlorothiazide

| Medium | 30 Minutes | 60 Minutes |
|---|---|---|
| pH 4.5 Acetate buffer USP | 91 | 97 |
| 0.1N HCl | 100 | 100 |
| pH 2.1 SGF | 88 | 98 |
| pH 6.8 Phosphate buffer USP | 82 | 85 |

Capsules prepared above are stored in closed HDPE bottles at 40° C. and 75% relative humidity for 6 months, and are analyzed for drug content and the levels of drug-related impurities. The results are shown below, wherein the amounts are percentages of the label content of the related active ingredient.

Assay

| Drug | Initial | 6 Months |
|---|---|---|
| Lisinopril | 97.8 | 99.0 |
| Hydrochlorothiazide | 99.5 | 99.3 |
| Simvastatin | 97.6 | 96.9 |
| Aspirin | 101.2 | 101.3 |

Impurities

| Drug | Impurity | Initial | 6 Months |
|---|---|---|---|
| Lisinopril | Monoacetyl | ND | ND |
| | SSS DKP | 0.05 | 0.33 |
| | RSS isomer | 0.09 | 0.09 |
| Hydrochlorothiazide | DSA | 0.04 | 0.07 |
| | CTZ | 0.04 | 0.04 |
| Simvastatin | Hydroxy acid | 0.26 | 0.25 |
| | Lovastatin | 0.23 | 0.21 |
| | Acetate ester | ND | ND |
| | Dehydro | 0.07 | 0.11 |
| | Dimer | 0.14 | 0.13 |
| Aspirin | Salicylic acid | 0.13 | 0.31 |

ND = Below the limit of detection.

The formulation is considered to have commercially acceptable storage stability.

1. A pharmaceutical formulation in the form of a capsule containing at least one tablet and comprising the active agents:
a cholesterol lowering agent;
aspirin;
an inhibitor of the renin-angiotensin system; and
a beta-adrenergic receptor blocking agent or a diuretic;
the active agents, or combinations of two or more thereof, being present in tablets, capsules, or as particles; optionally together with one or more tablets that do not contain any active agent; wherein a cholesterol lowering agent is physically separated from aspirin.

2. The pharmaceutical formulation of claim 1, wherein a capsule contains:
a) a tablet comprising a cholesterol-lowering agent, which is a HMG CoA reductase inhibitor;
b) a tablet comprising aspirin; and
c) a tablet comprising:
i) an inhibitor of the renin-angiotensin system, which an angiotensin converting enzyme inhibitor or an angiotensin receptor blocker, and
ii) a beta-adrenergic receptor blocking agent or a diuretic;
wherein one or more tablets is optionally coated.

3. (canceled)

4. The pharmaceutical formulation of claim 1, wherein a HMG CoA reductase inhibitor is lovastatin, atorvastatin, simvastatin, fluvastatin, rosuvastatin, or pravastatin.

5. (canceled)

6. The pharmaceutical formulation of claim 1, wherein an angiotensin converting enzyme inhibitor is enalapril, lisinopril, perindopril, trandolapril, or ramipril.

7. The pharmaceutical formulation of claim 1, wherein an angiotensin receptor blocker is losartan, irbesartan, olmesartan, candesartan, valsartan, or telmisartan.

8. The pharmaceutical formulation of claim 1, wherein a β-adrenergic receptor antagonist is atenolol, metoprolol, or propranolol.

9. The pharmaceutical formulation of 1, wherein a diuretic is chlorothiazide, hydrochlorothiazide, or indapamide.

10-11. (canceled)

12. A pharmaceutical formulation, in the form of a capsule comprising:
a) a tablet comprising simvastatin;
b) a tablet comprising aspirin; and
c) a tablet comprising lisinopril and either of atenolol or hydrochlorothiazide;
wherein one or more tablets is optionally coated.

13-27. (canceled)

28. The pharmaceutical formulation of claim 1, comprising:
a) a capsule, tablet, or particles containing aspirin; and
b) one or more of capsules, tablets, and particles, together containing simvastatin, lisinopril, and either hydrochlorothiazide or atenolol;
wherein only one of a) and b) contains one or more drugs in the form of particles.

29. (canceled)

30. The pharmaceutical formulation of claim 1, wherein any individual impurity related to an active agent is present in amounts less than about 5 percent of the label content of the active agent, and the total impurities related to active agents are present in amounts less than about 7 percent of the label content of the active agents.

31. The pharmaceutical formulation of claim 12, wherein: none of simvastatin-related impurities hydroxyl acid, lovastatin, acetate ester, dehydro simvastatin, and simvastatin dimer is present in amounts greater than about 1 percent of the label simvastatin content; or a salicylic acid impurity is not present in amounts greater than about 3 percent of the label aspirin content; or none of lisinopril-related impurities mono-acetyl lisinopril, RSS isomer of lisinopril, and SSS DPK is present in amounts greater than about 1 percent of the label lisinopril content; or none of the atenolol-related impurities o-monoacetyl atenolol, N-monoacetyl atenolol, diacetyl atenolol, and diol is present in amounts greater than about 1 percent of the label atenolol content; or neither of the hydrochlorothiazide-related impurities chlorothiazide and DSA is present in amounts greater than about 0.2 percent of the label hydrochlorothiazide content.

32-38. (canceled)

39. The pharmaceutical formulation of claim 12, wherein an aspirin tablet is enteric-coated.

40. The pharmaceutical formulation of claim 2, wherein an HMG CoA reductase inhibitor is lovastatin, atorvastatin, simvastatin, fluvastatin, rosuvastatin, or pravastatin.

41. The pharmaceutical formulation of claim 2, wherein an angiotensin converting enzyme inhibitor is enalapril, lisinopril, perindopril, trandolapril, or ramipril.

42. The pharmaceutical formulation of claim 2, wherein a β-adrenergic receptor antagonist is atenolol, metoprolol, or propranolol.

43. The pharmaceutical formulation of claim 2, wherein a diuretic is chlorothiazide, hydrochlorothiazide, or indapamide.
